(19) United States
(12) Patent Application Publication
(10) Pub. No.: US 2025/0179475 A1
(43) Pub. Date: Jun. 5, 2025

(54) METHODS FOR REDUCING CAPTURE OF ANALYTES

(71) Applicant: 10x Genomics, Inc., Pleasanton, CA (US)

(72) Inventors: Erik Leonard Henrik Borgstrom, Årsta (SE); Marco Mignardi, Sundbyberg (SE)

(21) Appl. No.: 19/044,080

(22) Filed: Feb. 3, 2025

Related U.S. Application Data
(63) Continuation of application No. PCT/US2023/029938, filed on Aug. 10, 2023.
(60) Provisional application No. 63/397,559, filed on Aug. 12, 2022.

(51) Int. Cl. C12N 15/10; G01N 33/68

(52) U.S. Cl. CPC C12N 15/1065; G01N 33/6845

(57) ABSTRACT

Provided herein are methods, compositions, and kits for reducing capture of analytes from a biological sample on a spatial array on areas not covered by a biological sample.

### CROSS-REFERENCE TO RELATED APPLICACTIONS

The Pursuant to 35 U.S.C. § 119(e), this application is a continuation of International Application PCT/US2023/029938, with an international filing date of Aug. 10, 2023, which claims the benefit of priority to U.S. Provisional Patent Application No. 63/397,559, filed on Aug. 12, 2022, the content of which is incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Unwanted biological interactions that can occur during experiments, such as background noise, non-specific interactions, and other unwanted interactions increase expense through wasted resources and time. In particular, spatial array based technologies benefit from precise determination of the location of analytes within a biological sample. However, some techniques for studying spatial heterogeneity in biological samples can cause analytes (e.g., nucleic acid analytes) from the biological sample to mislocalize to areas adjacent to (e.g., outside) the biological sample and be captured in such areas on the array. The result of capturing analytes on areas adjacent to the biological sample on the array (e.g., areas that do not correlate with the biological sample) can lead to a decrease in resolution, wasted resources, such as unnecessary costs attributed to sequencing (e.g., next generation sequencing) and inaccurate results. Thus, methods are needed to improve the incidence of captured analytes on areas associated with the biological sample and not in areas adjacent to the biological sample. The present disclosure features a solution to the problem of mislocalized analyte capture, wherein bulky moieties coupled to blocking reagents in the areas adjacent to a biological sample can improve efficiency, resource conservation, and resolution of spatial analysis.

### SUMMARY

The present disclosure provides for a method to block capture probes on a spatial array that are not directly under the biological sample e.g., in an area surrounding the biological sample, not covered by the biological sample, adjacent to the biological sample, etc. The methods described herein can mitigate analyte mislocalization and provide an improvement in resource conservation and a reduction and/or elimination of non-specific binding of analytes to unintended portions of the spatial array during performance of any of the methods described herein e.g., for determining a location of a target analyte in a biological sample.

Provided herein are methods for reducing capture of a target analyte in an area of an array not covered by a biological sample, the method including: disposing the biological sample on an array at a first area, where the array includes a plurality of capture probes, where: the first area includes a capture probe of the plurality of capture probes including (i) a spatial barcode and (ii) a capture domain, and a second area includes a capture probe of the plurality of capture probes including (i) a spatial barcode and (ii) a capture domain, and the second area is an area of the array not covered by the biological sample disposed on the array; contacting the second area of the array with a bulky moiety coupled to a blocking reagent, where the blocking reagent inhibits the capture probe in the second area from hybridizing to the target analyte; and permeabilizing the biological sample, such that the capture domain of the capture probe of the first area hybridizes to the target analyte, thereby reducing capture of the target analyte in the second area of the array not covered by the biological sample.

In some embodiments, the capture probe of the first area, the capture probe of the second area, or both, include one or more functional domains, a cleavage domain, a unique molecular identifier, and combinations thereof.

In some embodiments, the array includes one or more features, where the one or more features includes a bead, an inkjet spot, a masked spot, a well, or a hydrogel pad.

In some embodiments, the bulky moiety includes a magnetic bead, a high molecular weight protein or a polymer. In some embodiments, the polymer includes a polyethylene glycol (PEG) having a molecular weight of about 3000 g/mol to about 10,000 g/mol.

In some embodiments, the blocking reagent includes an enzyme. In some embodiments, the enzyme is a nuclease. In some embodiments, the nuclease is one or more of a DNase or a uracil DNA glycosylase. In some embodiments, the blocking reagent includes a 3′ OH disabling agent or a chemical cleavage agent.

In some embodiments, the method includes, prior to step (b), fixing the biological sample. In some embodiments, the step of fixing the biological sample includes the use of a fixative selected from the group consisting of: ethanol, methanol, acetone, formaldehyde, paraformaldehyde-Triton, glutaraldehyde, and combinations thereof.

In some embodiments, the method includes, prior to step (b), staining the biological sample. In some embodiments, the step of staining the biological sample includes the use of a biological stain selected from the group consisting of: acridine orange, Bismarck brown, carmine, Coomassie blue, cresyl violet, 4′,6-diamidino-2-phenylindole (DAPI), eosin, ethidium bromide, acid fuchsine, hematoxylin and eosin, Hoechst stains, iodine, methyl green, methylene blue, neutral red, Nile blue, Nile red, osmium tetroxide, propidium iodide, rhodamine, safranin, and combinations thereof. In some embodiments, the step of staining the biological sample includes the use of a detectable label selected from the group consisting of: a radioisotope, a fluorophore, a chemiluminescent compound, a bioluminescent compound, and combinations thereof.

In some embodiments, the biological sample is a tissue sample. In some embodiments, the tissue sample is a fixed tissue sample or a fresh frozen tissue sample. In some embodiments, the biological sample is a tissue section. In some embodiments, the fixed tissue section is a formalin-fixed paraffin-embedded tissue section, an acetone-fixed tissue section, a paraformaldehyde-fixed tissue section, or a methanol-fixed tissue section.

In some embodiments, the method includes a step (d) determining (i) the sequence corresponding to the spatial barcode of the capture probe of the first area of the array, or a complement thereof, and (ii) all or a portion of a sequence corresponding to the target analyte, or a complement thereof, and using the sequences of (i) and (ii) to determine the location of the target analyte in the biological sample. In some embodiments, the determining in step (d) includes sequencing (i) the spatial barcode of the capture probe of the first area of the array, or the complement thereof, and (ii) all or a portion of the target analyte, or the complement thereof. In some embodiments, the sequencing includes high-throughput sequencing. In some embodiments, the determining in step (d) includes extending a 3′ end of the capture probe of the first area of the array using the target analyte as a template.

In some embodiments, the method includes imaging the biological sample.

In some embodiments, the target analyte is genomic DNA or mRNA.

Also provided herein are methods for reducing capture of a target analyte in an area of an array not covered by a biological sample, the method including: (a) disposing the biological sample onto the array at a first area, where the array includes a plurality of capture probes, where: the first area includes a capture probe of the plurality of capture probes including a spatial barcode and a capture domain; and a second area includes a capture probe of the plurality of capture probes including a spatial barcode and a capture domain, the second area is an area of the array not covered by the biological sample disposed on the array; (b) contacting the second area of the array with a bulky moiety coupled to a blocking reagent, where the blocking reagent inhibits the capture probe in the second area from hybridizing to an analyte capture sequence of an analyte capture agent; (c) permeabilizing the biological sample, such that the capture domain of the capture probe of the first area of the array hybridizes to the analyte capture sequence; and (d) contacting a plurality of analyte capture agents with the permeabilized biological sample, where an analyte capture agent of the plurality of analyte capture agents includes an analyte binding moiety barcode, the analyte capture sequence, and an analyte binding moiety that binds to the target analyte, thereby reducing capture of the target analyte in the second area of the array not covered by the biological sample.

In some embodiments, the capture probe of the first area, the capture probe of the second area, or both, include one or more functional domains, a cleavage domain, a unique molecular identifier, and combinations thereof.

In some embodiments, the array includes one or more features including a bead, an inkjet spot, a masked spot, a well, or a hydrogel pad.

In some embodiments, the bulky moiety includes a high molecular weight protein or a polymer. In some embodiments, the polymer includes polyethylene glycol (PEG) of a molecular weight of about 3000 g/mol to about 10,000 g/mol.

In some embodiments, the blocking reagent includes an enzyme. In some embodiments, the enzyme includes a nuclease. In some embodiments, the nuclease includes one or both of a DNase and a uracil DNA glycosylase. In some embodiments, the blocking reagent includes a 3′ OH disabling agent or a chemical cleavage agent.

In some embodiments, the method includes, prior to step (b), fixing the biological sample. In some embodiments, the step of fixing the biological sample includes the use of a fixative selected from the group consisting of: ethanol, methanol, acetone, formaldehyde, paraformaldehyde-Triton, glutaraldehyde, and combinations thereof.

In some embodiments, the method includes, prior to step (b), staining the biological sample. In some embodiments, the step of staining the biological sample includes the use of a biological stain selected from the group consisting of: acridine orange, Bismarck brown, carmine, Coomassie blue, cresyl violet, 4′,6-diamidino-2-phenylindole (DAPI), eosin, ethidium bromide, acid fuchsine, hematoxylin and eosin, Hoechst stains, iodine, methyl green, methylene blue, neutral red, Nile blue, Nile red, osmium tetroxide, propidium iodide, rhodamine, safranin, and combinations thereof. In some embodiments, the step of staining the biological sample includes the use of a detectable label selected from the group consisting of: a radioisotope, a fluorophore, a chemiluminescent compound, a bioluminescent compound, and combinations thereof.

In some embodiments, the biological sample is a tissue sample. In some embodiments, the tissue sample is a fixed tissue sample or a fresh frozen tissue sample. In some embodiments, the biological sample is a fixed or fresh frozen tissue section. In some embodiments, the fixed tissue section is a formalin-fixed paraffin-embedded tissue section, an acetone-fixed tissue section, a paraformaldehyde-fixed tissue section, or a methanol-fixed tissue section.

In some embodiments, the method includes a step (e) determining (i) a sequence corresponding to the spatial barcode of the capture probe in the first area of the array, or a complement thereof, and (ii) a sequence corresponding to the analyte binding moiety barcode, or a complement thereof, and using the sequences of (i) and (ii) to determine the location of the target analyte in the biological sample. In some embodiments, the determining in step (d) includes sequencing (i) the spatial barcode of the capture probe of the first area of the array, or the complement thereof, and (ii) the analyte binding moiety barcode, or the complement thereof. In some embodiments, the sequencing includes high-throughput sequencing. In some embodiments, the determining in step (d) includes extending a 3′ end of the capture probe of the first area of the array using the analyte binding moiety barcode as a template.

In some embodiments, the method includes imaging the biological sample.

In some embodiments, the target analyte is a protein. In some embodiments, the protein is an intracellular protein, an extracellular protein or a cell surface protein.

In some embodiments, a second target analyte includes genomic DNA. In some embodiments, a second target analyte includes mRNA. In some embodiments, the analyte binding moiety includes an antibody or an antigen-binding fragment thereof.

Also provided herein are kits including: an array including a plurality of capture probes, where a capture probe of the plurality of capture probes includes a spatial barcode and a capture domain; and a bulky moiety coupled to a blocking reagent, where the blocking reagent inhibits the capture domain from hybridizing to a target analyte.

Also provided herein are kits including: an array including a plurality of capture probes, where a capture probe of the plurality of capture probes includes a spatial barcode and a capture domain; and a bulky moiety coupled to a blocking reagent, where the blocking reagent inhibits the capture domain from hybridizing to an analyte capture sequence.

In some embodiments, the kit includes a plurality of analyte capture agents, where an analyte capture agent of the plurality of analyte capture agents includes an analyte-binding moiety, an analyte-binding moiety barcode, and the analyte capture sequence. In some embodiments, the analyte-binding moiety barcode identifies the analyte-binding moiety. In some embodiments, the analyte-binding moiety is an antibody, or a fragment thereof.

In some embodiments, the kit includes one or more fixative(s). In some embodiments, the kit includes one or more biological stains, and optionally, where the one or more biological stains includes hematoxylin and eosin.

In some embodiments, the kit includes one or more permeabilization reagent(s) selected from the group consisting of: an organic solvent, a cross-linking agent, a detergent, an enzyme, and combinations thereof.

In some embodiments, the kit includes one or more of a reverse transcriptase, a terminal deoxynucleotidyl transferase, and a DNA polymerase.

In some embodiments, the kit includes a template switching oligonucleotide. In some embodiments, the kit includes a second strand primer. In some embodiments, the kit includes one or more adaptor(s).

Also provided herein are compositions, including an array having a first area and a second area, where the array includes a plurality of capture probes, where: the first area includes a capture probe of the plurality of capture probes including: (i) a spatial barcode and (ii) a capture domain bound to a target analyte from a biological sample; and a second area of the array includes a capture probe of the plurality of capture probes including: (i) a spatial barcode and (ii) a capture domain, where the capture probe of the plurality of capture probes in the second area is blocked by a blocking reagent coupled to a bulky moiety.

Also provided herein are compositions including an array having a first area and a second area, where the array includes a plurality of capture probes, where: the first area includes a capture probe of the plurality of capture probes including: (i) a spatial barcode and (ii) a capture domain bound to an analyte capture sequence; and a second area of the array includes a capture probe of the plurality of capture probes including: (i) a spatial barcode and (ii) a capture domain, where the capture probe of the plurality of capture in the second area is blocked by a blocking reagent coupled to a bulky moiety.

In some embodiments, the composition includes a plurality of analyte capture agents, where an analyte capture agent of the plurality of analyte capture agents includes an analyte-binding moiety, an analyte-binding moiety barcode, and the analyte capture sequence. In some embodiments, the analyte-binding moiety barcode identifies the analyte-binding moiety. In some embodiments, the analyte-binding moiety is an antibody, or a fragment thereof. In some embodiments, the analyte-binding moiety is bound to a protein.

In some embodiments, the array includes a biological sample that covers the first area of the array, but not the second area of the array.

In some embodiments, the capture probe of the first area, the capture probe of the second area, or both, include one or more functional domains, a cleavage domain, a unique molecular identifier, and combinations thereof.

In some embodiments, the array includes one or more features. In some embodiments, the one or more features includes a bead, an inkjet spot, a masked spot, a well, or a hydrogel pad.

In some embodiments, the bulky moiety includes a magnetic bead, a high molecular weight protein or a polymer. In some embodiments, the polymer includes polyethylene glycol (PEG) of a molecular weight of about 3000 g/mol to about 10,000 g/mol.

In some embodiments, the blocking reagent includes an enzyme. In some embodiments, the enzyme includes a nuclease. In some embodiments, the nuclease includes one or both of a DNase and a uracil DNA glycosylase. In some embodiments, the blocking reagent includes a 3′ OH disabling agent or a chemical cleavage agent.

In some embodiments, the biological sample is a tissue sample. In some embodiments, the tissue sample is a fixed tissue sample or a fresh frozen tissue sample. In some embodiments, the biological sample is a fixed or fresh frozen tissue section. In some embodiments, the fixed tissue section is a formalin-fixed paraffin-embedded tissue section, an acetone-fixed tissue section, a paraformaldehyde-fixed tissue section, or a methanol-fixed tissue section.

In some embodiments, the target analyte is DNA or mRNA.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 is a schematic diagram showing a biological sample on an array and an example of a bulky moiety coupled to a blocking reagent which is contacted with the array that is not associated with the biological sample (e.g., not inferior to the biological sample).

### DETAILED DESCRIPTION

Blocking one or more capture domains of capture probes on spatial arrays (or portions thereof) can increase efficiency and/or decrease non-specific binding of analytes on arrays (or portions thereof). In some examples, one or more capture probes (e.g., a capture domain of a capture probes) is blocked with a blocking reagent as described herein. A bulky moiety coupled to a blocking reagent can be contacted with the array (e.g., a second area of the array that surrounds a biological sample on the array), where the blocking reagent inhibits capture in the second area of the array e.g., prevents the capture domain of a capture probe from hybridizing to a target analyte.

Methods for reducing non-specific spatial interactions on a spatial array are described herein. The methods described herein can improve the resolution of spatial array results by reducing non-specific binding of targeted analytes. For example, the methods described herein can reduce non-specific binding of target analytes by capture probes (e.g., by blocking the capture domain of capture probes) not proximal to the analyte and/or associated with the biological sample (e.g., inferior to the biological sample). In some examples, analytes from a biological sample diffuse to areas of the array that are adjacent to the biological sample e.g., areas outside of the biological sample. This diffusion allows analytes to bind to the capture domain(s) of one or more capture probes that surround (e.g., outside) of the biological sample on the array. Non-specific binding increases background results (e.g., non-specific results), thereby decreasing resolution. Blocking the capture domain of capture probes surrounding the biological sample on the array can decrease such non-specific binding and thereby increase the resolution of the spatial results.

The methods described herein can also result in the conservation of resources. For example, the analysis of spatial arrays can include generating and sequencing a nucleic acid library. Binding of analytes to capture domains of capture probes outside of the biological sample can result in sequencing of undesired targets. Capture of analytes, or proxies thereof, can cause downstream sequencing inefficiencies. For example, a decrease in the amount of target analyte sequencing due to sequencing of captured analytes surrounding the biological sample is inefficient, reagent costly, and can result in a decrease is spatial resolution. The present disclosure provides solutions for minimizing or preventing capture of analytes outside or surrounding the biological sample.

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 1,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434): 1463-1467, 2019; Lee et al., Nat. Protoc. 10(3): 442-458, 2015; Trejo et al., PLOS ONE 14(2): e0212031, 2019; Chen et al., Science 348(6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits—Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663. Typically, a “barcode” is a label, or identifier, which conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, when the biological sample is fixed with a fixative including an alcohol (e.g., methanol or acetone-methanol mixture), it is not decrosslinked afterward. In some preferred embodiments, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments when a fixed frozen tissue sample is treated with a sucrose gradient, it can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used for antigen retrieval to decrosslink antigens and fixation medium in the biological sample. Thus, any suitable decrosslinking agent can be used in addition to or alternatively to citrate buffer. In some embodiments, for example, the biological sample (e.g., a fixed frozen tissue sample) is decrosslinked with TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, it is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, it can be rehydrated with an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1;9(10): 5188-96; Kap M. et al., PLOS One.; 6(11):e27704 (2011); and Mathieson W. et al., Am J Clin Pathol.; 146(1): 25-40 (2016), each of which are hereby incorporated by reference in their entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes as disclosed herein to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, the entire contents of which are incorporated herein by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II)(d)(ii) of PCT Publication No. WO2020/176788 and/or U.S. Patent Application Publication No. 2020/0277663.

In some instances, a capture probe and a nucleic acid analyte (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues in the other nucleic acid sequence. The complementary residues within a particular complementary nucleic acid sequence need not always be contiguous with each other and can be interrupted by one or more non-complementary residues within the complementary nucleic acid sequence. In some embodiments, at least 60%, but less than 100%, of the residues of one of the two complementary nucleic acid sequences are complementary to residues in the other nucleic acid sequence. In some embodiments, at least 70%, 80%, 90%, 95% or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence. In some embodiments, the biological sample is mounted on a first substrate and the substrate comprising the array of capture probes is a second substrate. During this process, one or more analytes or analyte derivatives (e.g., intermediate agents; e.g., ligation products) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

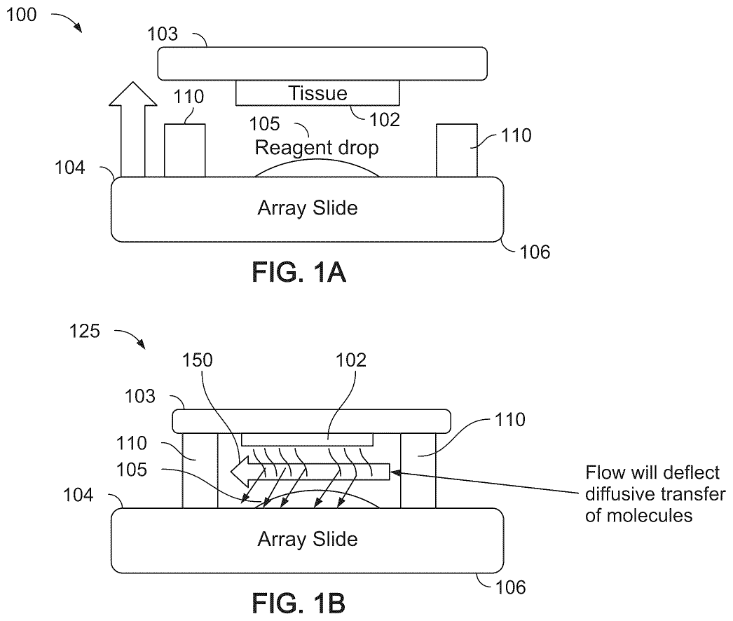

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2, each of which are incorporated by reference in their entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

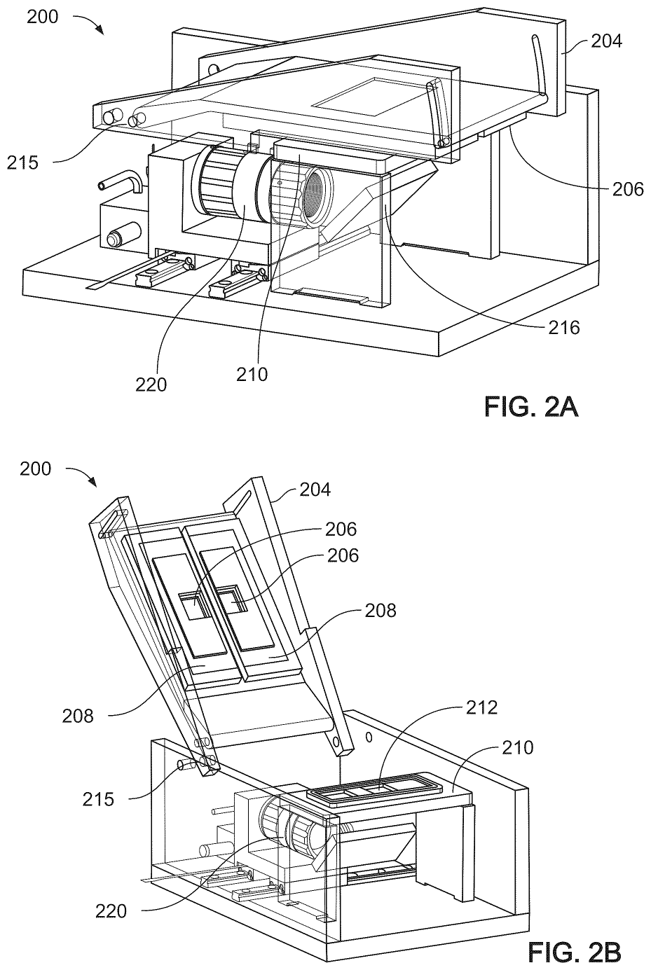

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

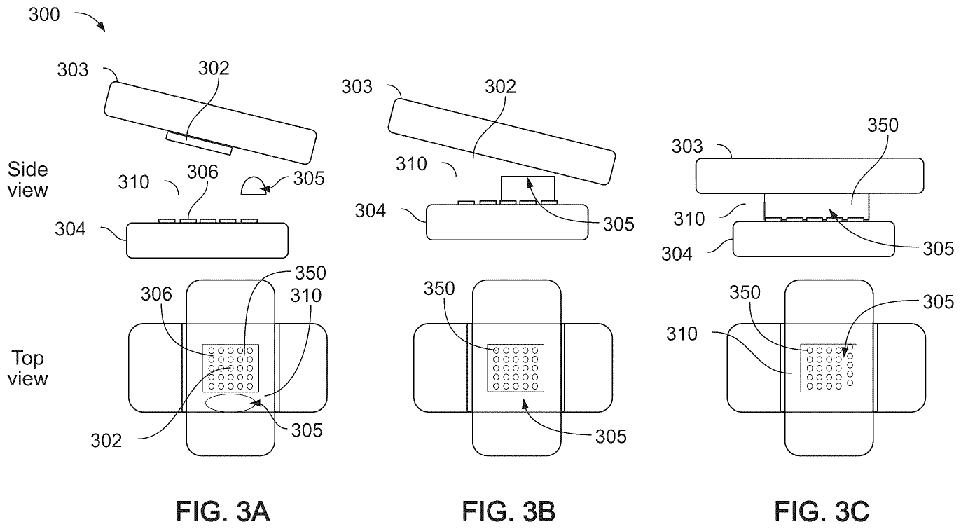

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the second substrate) may contact the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

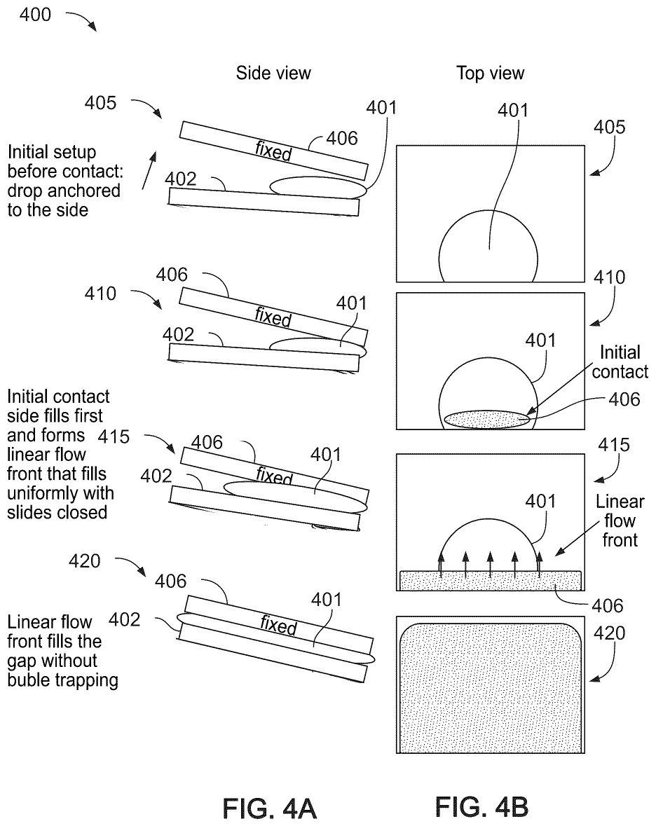

FIG. 4A is a side view of the angled closure workflow 400 in accordance with some exemplary implementations. FIG. 4B is a top view of the angled closure workflow 400 in accordance with some exemplary implementations. As shown at 405, reagent medium 401 is positioned to the side of the substrate 402 contacting the spring.

At step 410, the dropped side of the angled substrate 406 contacts the reagent medium 401 first. The contact of the substrate 406 with the reagent medium 401 may form a linear or low curvature flow front that fills uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side and creating a linear or low curvature flow front that may prevent or reduce bubble trapping between the substrates.

At step 420, the reagent medium 401 fills the gap between the substrate 406 and the substrate 402. The linear flow front of the liquid reagent may form by squeezing the 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of sodium dodecyl sulfate (SDS) or a sodium salt thereof, proteinase K, pepsin, N-lauroylsarcosine, and RNase.

In some embodiments, the reagent medium comprises polyethylene glycol (PEG). In some embodiments, the PEG is from about 2K to about 16K. In some embodiments, the PEG is 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In certain embodiments a dried permeabilization reagent is applied or formed as a layer on the first substrate or the second substrate or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended by a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663. Analysis of captured analytes (and/or intermediate agents or portions thereof), for example, including sample removal, extension of capture probes using the capture analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663. Some quality control measures are described in Section (II)(h) of PCT Publication No. WO2020/176788 and/or U.S. Patent Application Publication No. 2020/0277663.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up- and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663.

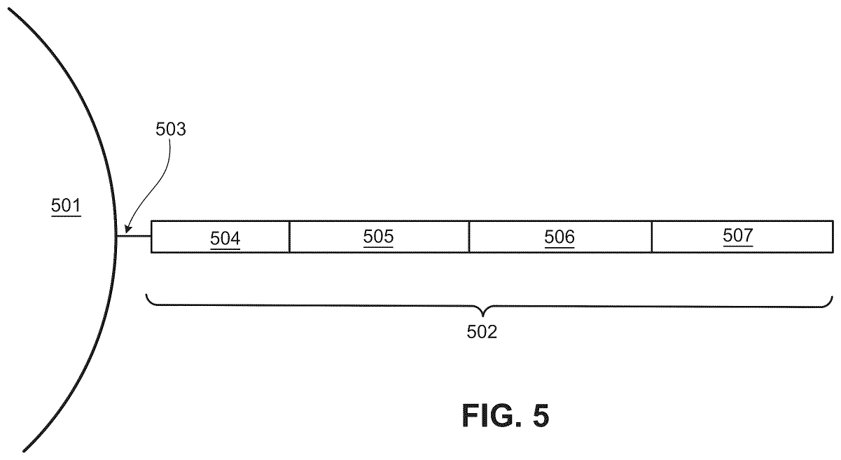

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to a analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

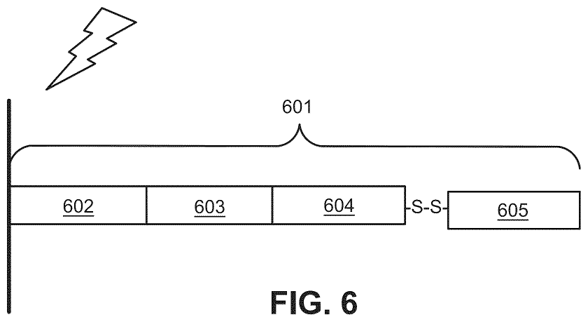

FIG. 6 is a schematic illustrating a cleavable capture probe, wherein the cleaved capture probe can enter into a non-permeabilized cell and bind to analytes within the sample. The capture probe 601 contains a cleavage domain 602, a cell penetrating peptide 603, a reporter molecule 604, and a disulfide bond (—S—S—). 605 represents all other parts of a capture probe, for example a spatial barcode and a capture domain.

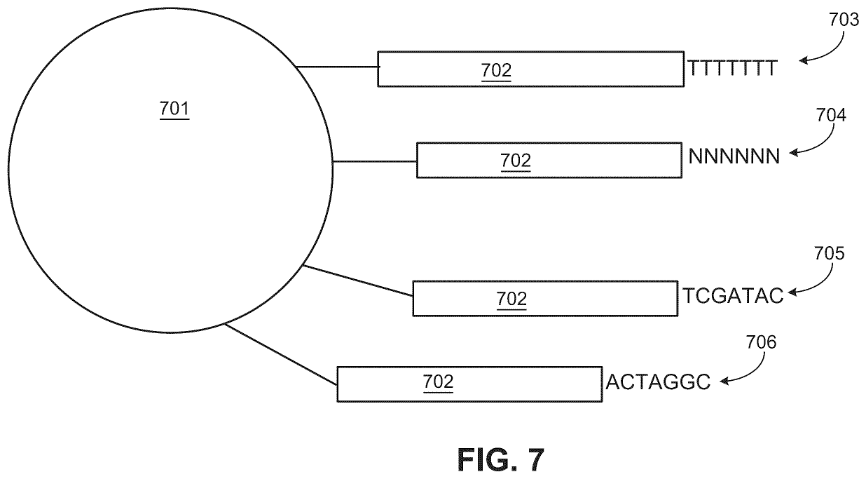

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature includes the spatial barcode 702 in combination with a poly(T) capture domain 703, designed to capture mRNA target analytes. A second type of capture probe associated with the feature includes the spatial barcode 702 in combination with a random N-mer capture domain 704 for gDNA analysis. A third type of capture probe associated with the feature includes the spatial barcode 702 in combination with a capture domain complementary to the analyte capture agent of interest 705. A fourth type of capture probe associated with the feature includes the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 are common to all of the probes attached to a given feature. In some embodiments, the UMI sequence 506 of a capture probe attached to a given feature is different from the UMI sequence of a different capture probe attached to the given feature.

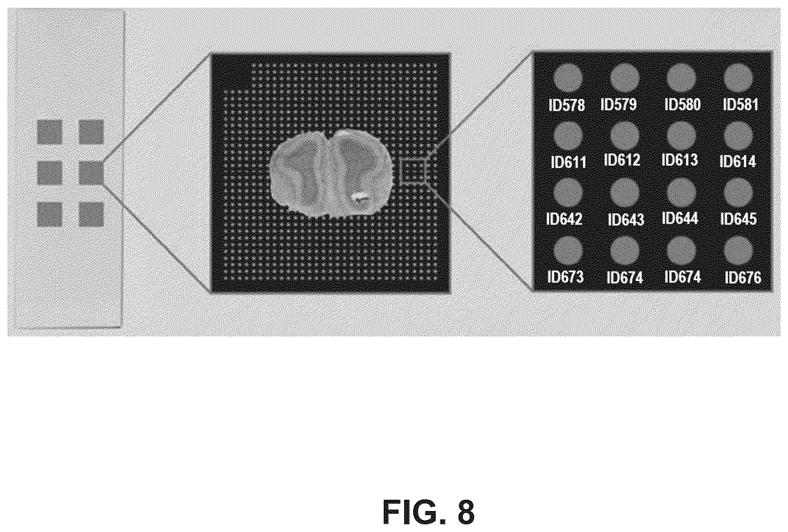

FIG. 8 depicts an exemplary arrangement of barcoded features within an array. From left to right, FIG. 8 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14): e128.Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., a T4 RNA ligase (Rn12), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location and optionally the abundance of the analyte in the biological sample.

In some instances, one or both of the oligonucleotides may hybridize to genomic DNA (gDNA) which can lead to false positive sequencing data from ligation events on gDNA (off target) in addition to the desired (on target) ligation events on target nucleic acids, (e.g., mRNA). Thus, in some embodiments, the disclosed methods can include contacting the biological sample with a deoxyribonuclease (DNase). The DNase can be an endonuclease or exonuclease. In some embodiments, the DNase digests single- and/or double-stranded DNA. Suitable DNases include, without limitation, a DNase I and a DNase II. Use of a DNase as described can mitigate false positive sequencing data from off target gDNA ligation events.

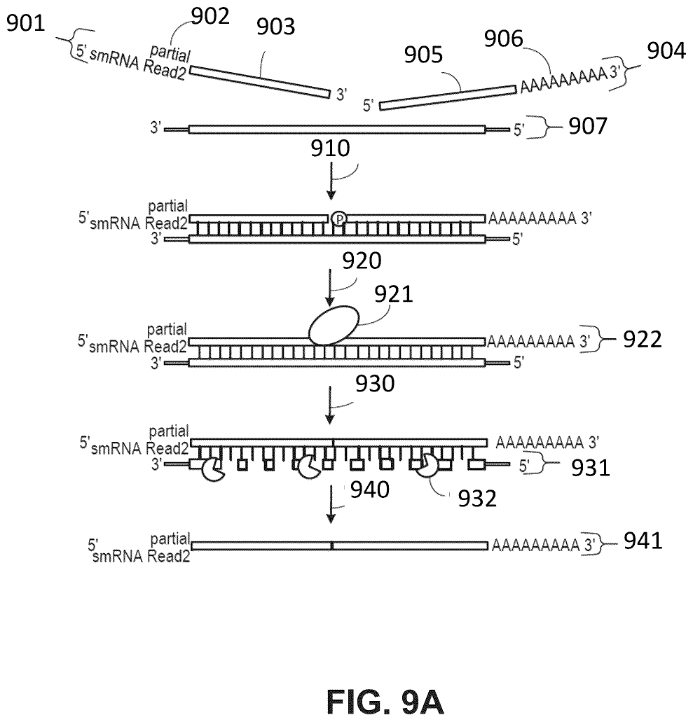

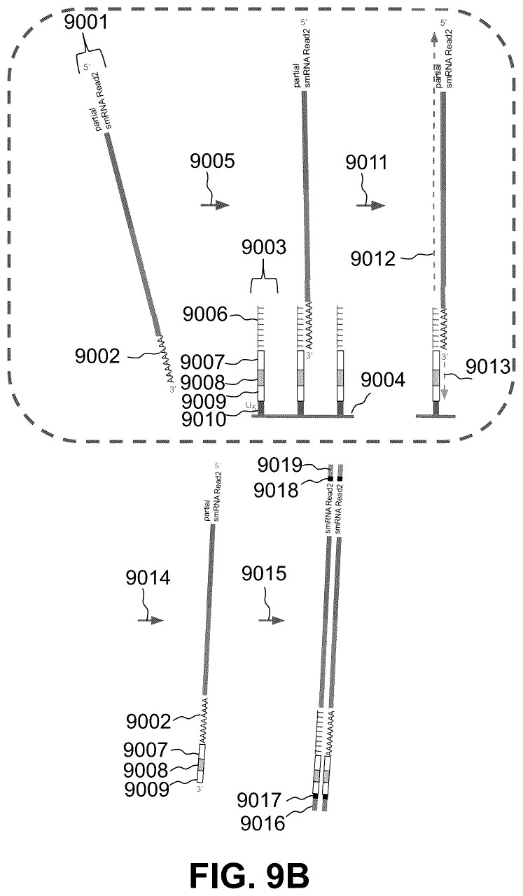

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly-A sequence) 906, the first probe 901 and a second probe 904 hybridize 910 to an analyte 907. A ligase 921 ligates 920 the first probe to the second probe thereby generating a ligation product 922. The ligation product is released 930 from the analyte 931 by digesting the analyte using an endoribonuclease 932. The sample is permeabilized 940 and the ligation product 941 is able to hybridize to a capture probe on the substrate. Methods and composition for spatial detection using templated ligation have been described in PCT Publ. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, reverse transcription (RT) reagents can be added to permeabilized biological samples. Incubation with the RT reagents can extend the capture probes 9011 to produce spatially-barcoded full-length cDNA 9012 and 9013 from the captured ligation products.

In some embodiments, the extended ligation products can be denatured 9014 from the capture probe and transferred (e.g., to a clean tube) for amplification, and/or library construction. The spatially-barcoded ligation products can be amplified 9015 via PCR prior to library construction. P5 9016, i5 9017, i7 9018, and P7 9019, and can be used as sequencing probes (P5 and P7) and sample indexes (i5 and i7) when utilizing Illumina sequencing instruments. The amplicons can then be sequenced using paired-end sequencing using TruSeq Read 1 and TruSeq Read 2 as sequencing primer sites. Depending on the sequencing instrument and technology used, probes, indexes and primer sequencing sites may be different, but can equally be incorporated into the methods disclosed herein.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663.

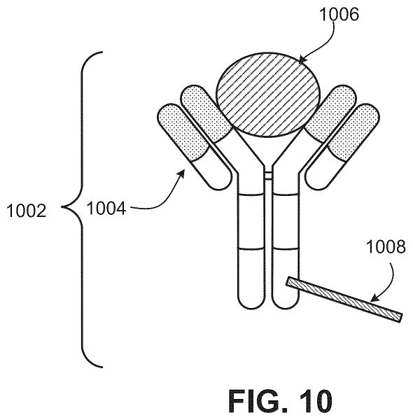

FIG. 10 is a schematic diagram of an exemplary analyte capture agent 1002 comprised of an analyte-binding moiety 1004 and an analyte-binding moiety barcode domain 1008. The exemplary analyte-binding moiety 1004 is a molecule capable of binding to an analyte 1006 and the analyte capture agent is capable of interacting with a spatially-barcoded capture probe. The analyte-binding moiety can bind to the analyte 1006 with high affinity and/or with high specificity. The analyte capture agent can include an analyte-binding moiety barcode domain 1008 which serves to identify the analyte binding moiety, and a capture domain which can hybridize to at least a portion or an entirety of a capture domain of a capture probe. The analyte-binding moiety 1004 can include a polypeptide and/or an aptamer. The analyte-binding moiety 1004 can include an antibody or antibody fragment (e.g., an antigen-binding fragment).

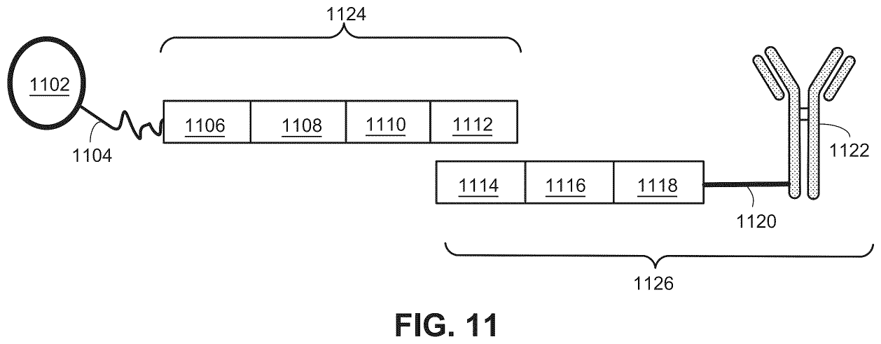

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequences 1106 and a UMI 1110, as described elsewhere herein. The capture probe can be affixed 1104 to a feature such as a bead 1102. The capture probe can also include a capture domain 1112 that is capable of binding to an analyte capture agent 1126. The analyte-binding moiety barcode domain of the analyte capture agent 1126 can include a functional sequence 1118, analyte binding moiety barcode 1116, and an analyte capture sequence 1114 that is capable of binding (e.g., hybridizing) to the capture domain 1112 of the capture probe 1124. The analyte capture agent can also include a linker 1120 that allows the analyte-binding moiety barcode domain (e.g., including the functional sequence 1118, analyte binding barcode 1116, and analyte capture sequence 1114) to couple to the analyte binding moiety 1122. In some embodiments, the linker is a cleavable linker. In some embodiments, the cleavable linker is a photo-cleavable linker, a UV-cleavable linker, or an enzyme cleavable linker. In some instances, the cleavable linker is a disulfide linker. A disulfide linker can be cleaved by use of a reducing agent, such as dithiothreitol (DTT), Beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022).

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Methods for Reducing Capture of Target Analytes on an Area of the Array Not Covered by a Biological Sample

Spatial tissue arrays allow researchers insight into a multitude of cellular activity to identify gene expression, protein locations, and other cellular activity tracking within the spatial context of a tissue. The benefits of correlating spatial biological relationships with diseases and disorders does, and will, continue to advance many fields of scientific study. However, improvements in the resolution of spatial relationships between the cellular activities and diseases and disorders would enhance those data. For example, when a biological sample (e.g., a tissue section) affixed to a spatial array slide is permeabilized, analytes are released from the biological sample and can, via diffusion, move to areas of the array not covered by the biological sample (e.g., tissue section), for example to areas surrounding or outside the biological sample, where non-specific spatial analyte capture can occur. This type of spatial analyte capture can decrease the resolution of the desired spatial analyte data since the analyte is correlated with a spatial location (e.g., via the spatial barcode) outside of the biological sample. Further, capture of target analytes outside of the biological sample causes downstream sequencing inefficiencies including a decrease in the amount of target analyte sequencing due to sequencing of non-specific captured analytes. Such capture is inefficient, uninformative, and reagent costly. The present disclosure provides solutions for minimizing and/or preventing capture of analytes on an area of the array not covered by the biological sample.

Provided herein are methods for reducing capture of a target analyte in an area of an array not covered by a biological sample, the method including: (a) disposing a non-permeabilized biological sample on an array at a first area, where the array includes a plurality of capture probes, where: (i) the first area includes a capture probe of the plurality of capture probes including (i) a spatial barcode and (ii) a capture domain, and (ii) a second area includes a capture probe of the plurality of capture probes including (i) a spatial barcode and (ii) a capture domain, and the second area is an area of the array not covered by the non-permeabilized biological sample disposed on the array; (b) contacting the second area of the array with a bulky moiety coupled to a blocking reagent, where the bulky moiety coupled to the blocking reagent inhibits the capture probe in the second area from hybridizing to the target analyte; and (c) permeabilizing the biological sample, such that the capture domain of the capture probe of the first area hybridizes to the target analyte, thereby reducing capture of the target analyte in the second area of the array not covered by the biological sample.

Also provided herein are methods for reducing capture of a target analyte in an area of an array not covered by a biological sample, the method including: (a) disposing a non-permeabilized biological sample onto an array at a first area, where the array includes a plurality of capture probes, where: (i) the first area includes a capture probe of the plurality of capture probes including a spatial barcode and a capture domain; and (ii) a second area comprises a capture probe of the plurality of capture probes comprising (i) a spatial barcode and (ii) a capture domain, the second area is an area of the array not covered by the biological sample disposed on the array; (b) contacting the second area of the array with a bulky moiety coupled to a blocking reagent, where the bulky moiety coupled to the blocking reagent inhibits the capture probe in the second area from hybridizing to an analyte capture sequence; (c) permeabilizing the biological sample, such that the capture domain of the capture probe of the first area of the array hybridizes to the analyte capture sequence; and (d) contacting a plurality of analyte capture agents with the permeabilized biological sample, where an analyte capture agent of the plurality of analyte capture agents includes an analyte binding moiety barcode, the analyte capture sequence, and an analyte binding moiety that binds to the target analyte, thereby reducing capture of the target analyte in the second area of the array not covered by the biological sample.

In addition to capturing intermediate products such as analyte capture agents, the methods provided herein also include capturing intermediate products such as ligation products (as described herein).

As used herein a “bulky moiety” is a molecule that is applied to the array such that it anchors a coupled blocking reagent to the location where it was delivered. For example, once placed at a location on the array the bulky moiety stays positioned at the location to where it was delivered, thereby localizing the blocking reagent.

In some embodiments, the bulky moiety is a high molecular weight protein. A “high molecular weight protein” has a molecular weight of at least about 300 kilodalton (kDa). In some embodiments, the high molecular weight protein is about 300 kDa to about 1200 kDa. For example, the high molecular weight protein can have a molecular weight of at least about 100 kDa, at least about 200 kDa, at least about 300 kDa, at least about 400 kDa, at least about 500 kDa, at least about 600 kDa, at least about 700 kDa, at least about 800 kDa, at least about 900 kDa, at least about 1000 kDa, or at least about 1200 kDa or more.

In some embodiments, the bulky moiety comprises a polymer. In some embodiments, the polymer comprises polyethylene glycol (PEG). In some embodiments, the PEG has a molecular weight of about 1000 g/mol to about 20000 g/mol. In some embodiments, the PEG has a molecular weight of about 2000 g/mol to about 15000 g/mol. In some embodiments, the PEG has a molecular weight of about 3000 g/mol to about 10000 g/mol. In some embodiments, the PEG has a molecular weight of at least about 1000 g/mol, at least about 2000 g/mol, at least about 3000 g/mol, at least about 4000 g/mol, at least about 5000 g/mol, at least about 6000 g/mol, at least about 7000 g/mol, at least about 8000 g/mol, at least about 9000 g/mol, at least about 10000 g/mol, at least about 11000 g/mol, at least about 12000 g/mol, at least about 13000 g/mol, at least about 14000 g/mol, at least about 15000 g/mol, at least about 16000 g/mol, at least about 17000 g/mol, at least about 18000 g/mol, at least about 19000 g/mol, or at least about 20,000 g/mol.

As used herein a “blocking reagent” is a molecule that prevent or inhibits the capture domain of a capture probe from hybridizing to an analyte or an analyte capture sequence and/or removes the capture probe from the array (e.g., in the second area of the array) altogether. In some embodiments, the blocking reagent is an enzyme. In some embodiments, the enzyme is DNase (e.g., any suitable DNase). In some embodiments, the enzyme is uracil DNA glycosylase. For example, the blocking reagent is coupled to the bulky moiety and delivered to the array where the bulky moiety stabilizes the blocking reagent in the position on the array to where it is delivered and the blocking reagent coupled to the bulky moiety can inhibit binding to the capture domain and/or remove and/or degrade the capture probe on the array. In some embodiments, the blocking reagent is a DNase and when delivered to the array (e.g., the second area of the array) the DNase can degrade the DNA-based capture probes thereby inhibiting capture of analytes or analyte capture sequences in areas surrounding the biological sample. In some embodiments, the capture probe includes a cleavage domain comprising a sequence that is recognized by one or more enzymes capable of cleaving a nucleic acid molecule, e.g., capable of breaking the phosphodiester linkage between two or more nucleotides. A bond can be cleavable via other nucleic acid molecule targeting enzymes, such as restriction enzymes (e.g., restriction endonucleases). For example, the cleavage domain can include a restriction endonuclease (restriction enzyme) recognition sequence. Restriction enzymes cut double-stranded or single stranded DNA at specific recognition nucleotide sequences known as restriction sites. In some embodiments, a rare-cutting restriction enzyme, e.g., enzymes with a long recognition site (at least 8 base pairs in length), is used to reduce the possibility of cleaving elsewhere in the capture probe. For example, when the blocking reagent is uracil DNA glycosylase the enzyme can cleave one or more uracil nucleic acids in the capture probe (e.g., a cleavage domain of the capture probe). In some embodiments, the cleavage domain includes a poly (U) sequence which can be cleaved by a mixture of Uracil DNA glycosylase (UDG) and the DNA glycosylase-lyase Endonuclease VIII, commercially known as the USER™ enzyme. In such embodiments, the capture probe is removed from the array (e.g., the second area of the array) and can be washed away.

In some embodiments, the blocking reagent is a cleavage agent. In some embodiments, the cleavage agent is a chemical cleavage domain. Other examples of cleavage domains include labile chemical bonds such as, but not limited to, ester linkages (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), an abasic or apurinic/apyrimidinic (AP) site (e.g., cleavable with an alkali or an AP endonuclease), or a phosphodiester linkage (e.g., cleavable via a nuclease (e.g., DNase)).

In some embodiments, the blocking reagent comprise a 3′ OH disabling agent. In some embodiments, the 3′ OH disabling agent is an exonuclease (e.g., any suitable exonuclease known in the art). In some embodiments, the exonuclease is a 3′ exonuclease. For example, a 3′ exonuclease can remove nucleotides from the 3′ hydroxyl terminus of the capture probe by cleavage of phosphodiester bonds via hydrolysis, thereby disabling the capture probe. In some embodiments, the 3′ OH disabling agent is a kinase. For example, the kinase (e.g., any suitable kinase known in the art) can phosphorylate the 3′ end of the capture probe, thereby disabling the capture probe from an extension reaction. In some embodiments, the 3′ OH disabling agent is a 3′ OH protecting group. Non-limiting examples of 3′ OH protecting groups are described e.g., in A. J. Camden, et al., DNA Oligonucleotide 3′-Phosphorylation by a DNA enzyme, Biochemistry, 55(18): 2671-2676 (2016) and Basic Principles of Organic Chemistry, John D Robert and Marjorie C. Caserio Eds, Chapter 15:10 LibreTexts (2022), both of which are incorporated herein by reference in their entireties.

In some embodiments, the bulky moiety and the blocking reagent are coupled. In some embodiments, the bulky moiety and blocking reagent are coupled directly (e.g., covalently or noncovalently). In some embodiments, the bulky moiety and the blocking reagent are coupled indirectly (e.g., via a linker). As used herein, a “linker” generally refers to a multifunctional (e.g., bifunctional, trifunctional) reagent used for conjugating two or more chemical moieties. In some embodiments, the linker is a benzophenone. In some embodiments, the linker is an amino methacrylamide. For example, the linker can be 3-aminopropyl methacrylamide. In some embodiments, the linker is a PEG linker. In some embodiments, the linker is a cleavable linker. Exemplary linkers are known in the art and examples of such can be found in WO 2020/176788, which is incorporated herein by reference in its entirety.

Attachment (coupling) of the bulky moiety and blocking reagent can be achieved through any of a variety of direct or indirect, covalent or non-covalent associations or attachments. For example, chemical conjugation techniques (e.g., Lightning-Link® antibody labelling kits available from Innova Biosciences) can be used. In some embodiments, a bulky moiety can be coupled to blocking reagent using non-covalent attachment mechanisms (e.g., using biotinylated bulky moieties that include one or more biotinylated linker(s), coupled to blocking reagents with an avidin or streptavidin linker.) Biotinylation techniques can be used, and are described for example in Fang et al., Nucleic Acids Res. (2003), 31(2): 708-715, the entire contents of which are incorporated by reference herein. Likewise, protein and peptide biotinylation techniques have been developed and can be used, and are described for example in U.S. Pat. No. 6,265,552, the entire contents of which are incorporated by reference herein. Furthermore, click reaction chemistry such as a methyltetrazine-PEG5-NHS ester reaction, a TCO-PEG4-NHS ester reaction, or the like, can be used to couple bulky moieties to blocking reagents. The reactive moiety on the bulky moiety can also include amine for targeting aldehydes, amine for targeting maleimide (e.g., free thiols), azide for targeting click chemistry compounds (e.g., alkynes), biotin for targeting streptavidin, phosphates for targeting EDC, which in turn targets active ester (e.g., NH2). The reactive moiety on the bulky moiety can be a chemical compound or group that binds to the reactive moiety on the blocking reagent. Exemplary strategies to conjugate the bulky moiety to the blocking reagent include the use of commercial kits (e.g., Solulink, Thunder link), conjugation of mild reduction of hinge region and maleimide labelling, stain-promoted click chemistry reaction to labeled amides (e.g., copper-free), and conjugation of periodate oxidation of sugar chain and amine conjugation. In some embodiments, certain steps (e.g., COOH activation (e.g., EDC) and homobifunctional cross linkers) can be avoided to prevent the bulky moieties from conjugating to themselves.

The biological sample can be any of the biological samples described herein. For example, in some embodiments, the biological sample is a tissue sample (e.g., a tissue section). In other embodiments, the biological sample is a clinical sample (e.g., whole blood, blood-derived products, blood cells, cultured tissue, cultured cells, or a cell suspension). In some embodiments, the biological sample is an organoid, embryonic stem cells, pluripotent stem cells, or any combination thereof. Non-limiting examples of an organoid include a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, a retinal organoid, or any combination thereof. In other example embodiments, the biological sample can include diseased cells, fetal cells, immune cells, cellular macromolecules, organelles, extracellular polynucleotides, or any combination thereof.

Non-limiting examples of target analytes include nucleic acids such as DNA or RNA. Non-limiting examples of DNA analytes include genomic DNA, methylated DNA, specific methylated DNA sequences, fragmented DNA, mitochondrial DNA, in situ synthesized PCR products, and viral DNA.

Non-limiting examples RNA analytes include various types of coding and non-coding RNA. Examples of the different types of RNA analytes include messenger RNA (mRNA), ribosomal RNA (rRNA), transfer RNA (RNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). The RNA can be from an RNA virus, for example RNA viruses from Group III, IV or V of the Baltimore classification system. The RNA can be from a retrovirus, such as a virus from Group VI of the Baltimore classification system.

In some embodiments, the present disclosure features methods of reducing capture of a second target analyte in addition to a first target analyte. In some embodiments, the second analyte is genomic DNA. In some embodiments, the second target analyte is mRNA.

In some embodiments, the biological sample can be fixed (e.g., between steps (a) and (b) the biological sample can be fixed using any of the techniques described herein or known in the art). In some embodiments, fixing the biological sample comprises the use of a fixative selected from the group of ethanol, methanol, acetone, formaldehyde, formalin, paraformaldehyde-Triton, glutaraldehyde, or any combination thereof. In some embodiments, a fixed biological sample is a formalin-fixed paraffin-embedded tissue sample.

In some embodiments, the non-permeabilized biological sample can be stained. In some embodiments, the staining includes optical labels as described herein, including, but not limited to, fluorescent (e.g., fluorophore), radioactive (e.g., radioisotope), chemiluminescent (e.g., a chemiluminescent compound), a bioluminescent compound, calorimetric, or colorimetric detectable labels. In some embodiments, the staining includes a fluorescent antibody directed to a target analyte (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain, such as hematoxylin and eosin (H&E) or periodic acid-schiff (PAS). In some embodiments, staining the biological sample comprises the use of a biological stain including, but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, significant time (e.g., days, months, or years) can elapse between staining and/or imaging the biological sample.

### First and Second Areas

In some embodiments of any of the methods described herein, an array can have a first area upon which is disposed a biological sample and a second area that is adjacent to the biological sample. For instance, some embodiments of any of the methods described herein include disposing a biological sample (e.g., a non-permeabilized biological sample) onto an array (e.g., any of the exemplary arrays described herein), where the array then has a first area covered by the non-permeabilized biological sample and a second area not covered by the non-permeabilized biological sample.

In some examples, the first area can represent a portion of the array that is covered by the biological sample, e.g., about 10% to about 99%, about 10% to about 95%, about 10% to about 90%, about 10% to about 85%, about 10% to about 80%, about 10% to about 75%, about 10% to about 70%, about a 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 99%, about 15% to about 95%, about 15% to about 90%, about 15% to about 85%, about 15% to about 80%, about 15% to about 75%, about 15% to about 70%, about a 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 99%, about 20% to about 95%, about 20% to about 90%, about 20% to about 85%, about 20% to about 80%, about 20% to about 75%, about 20% to about 70%, about a 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 99%, about 25% to about 95%, about 25% to about 90%, about 25% to about 85%, about 25% to about 80%, about 25% to about 75%, about 25% to about 70%, about a 25% to about 65%, about 25% to about 60%, about 25% to about 55%, about 25% to about 50%, about 25% to about 45%, about 25% to about 40%, about 25% to about 35%, about 25% to about 30%, about 30% to about 99%, about 30% to about 95%, about 30% to about 90%, about 30% to about 85%, about 30% to about 80%, about 30% to about 75%, about 30% to about 70%, about a 30% to about 65%, about 30% to about 60%, about 30% to about 55%, about 30% to about 50%, about 30% to about 45%, about 30% to about 40%, about 30% to about 35%, about 35% to about 99%, about 35% to about 95%, about 35% to about 90%, about 35% to about 85%, about 35% to about 80%, about 35% to about 75%, about 35% to about 70%, about a 35% to about 65%, about 35% to about 60%, about 35% to about 55%, about 35% to about 50%, about 35% to about 45%, about 35% to about 40%, about 40% to about 99%, about 40% to about 95%, about 40% to about 90%, about 40% to about 85%, about 40% to about 80%, about 40% to about 75%, about 40% to about 70%, about a 40% to about 65%, about 40% to about 60%, about 40% to about 55%, about 40% to about 50%, about 40% to about 45%, about 45% to about 99%, about 45% to about 95%, about 45% to about 90%, about 45% to about 85%, about 45% to about 80%, about 45% to about 75%, about 45% to about 70%, about a 45% to about 65%, about 45% to about 60%, about 45% to about 55%, about 45% to about 50%, about 50% to about 99%, about 50% to about 95%, about 50% to about 90%, about 50% to about 85%, about 50% to about 80%, about 50% to about 75%, about 50% to about 70%, about a 50% to about 65%, about 50% to about 60%, about 50% to about 55%, about 55% to about 99%, about 55% to about 95%, about 55% to about 90%, about 55% to about 85%, about 55% to about 80%, about 55% to about 75%, about 55% to about 70%, about a 55% to about 65%, about 55% to about 60%, about 60% to about 99%, about 60% to about 95%, about 60% to about 90%, about 60% to about 85%, about 60% to about 80%, about 60% to about 75%, about 60% to about 70%, about a 60% to about 65%, about 65% to about 99%, about 65% to about 95%, about 65% to about 90%, about 65% to about 85%, about 65% to about 80%, about 65% to about 75%, about 65% to about 70%, about 70% to about 99%, about 70% to about 95%, about 70% to about 90%, about 70% to about 85%, about 70% to about 80%, about 70% to about 75%, about 75% to about 99%, about 75% to about 95%, about 75% to about 90%, about 75% to about 85%, about 75% to about 80%, about 80% to about 99%, about 80% to about 95%, about 80% to about 90%, about 80% to about 85%, about 85% to about 99%, about 85% to about 95%, about 85% to about 90%, about 90% to about 99%, about 90% to about 95%, or about 95% to about 99%, of the total area of the array covered by the biological sample.

The second area represents a portion of the array that is not covered by the biological sample.

In some embodiments, the second area of the array can be contacted by the solution for, e.g., about 5 minutes to about 1 hour, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 1 hour, about 10 minutes to about 50 minutes, about 10 minutes to about 40 minutes, about 10 minutes to about 30 minutes, about 10 minutes to about 20 minutes, about 20 minutes to about 1 hour, about 20 minutes to about 50 minutes, about 20 minutes to about 40 minutes, about 20 minutes to about 30 minutes, about 30 minutes to about 1 hour, about 30 minutes to about 50 minutes, about 30 minutes to about 40 minutes, about 40 minutes to about 1 hour, about 40 minutes to about 50 minutes, or about 50 minutes to about 1 hour, at a temperature of about 4° C. to about 35° C., about 4° C. to about 30° C., about 4° C. to about 25° C., about 4° C. to about 20° C., about 4° C. to about 15° C., about 4° C. to about 10° C., about 10° C. to about 35° C., about 10° C. to about 30° C., about 10° C. to about 25° C., about 10° C. to about 20° C., about 10° C. to about 15° C., about 15° C. to about 35° C., about 15° C. to about 30° C., about 15° C. to about 25° C., about 15° C. to about 20° C., about 20° C. to about 35° C., about 20° C. to about 30° C., about 20° C. to about 25° C., about 25° C. to about 35° C., about 25° C. to about 30° C., or about 30° C. to about 35° C.

The resulting cDNA from the captured analytes or proxies thereof (e.g., a ligation product, an analyte capture sequence) can be denatured from the capture probe template and transferred (e.g., to a clean tube) for amplification, and/or library construction as described herein. The spatially-barcoded, full-length cDNA(s) can be amplified via PCR prior to library construction. The cDNA can then be enzymatically fragmented and size-selected in order to optimize the cDNA amplicon size. P5, P7, i7, and i5 can be incorporated into the library as for downstream sequencing, and additional library sequencing regions, such as TruSeq Read 2, can be added via End Repair, A-tailing, Adaptor Ligation, and PCR. The cDNA fragments can then be sequenced using paired-end sequencing using TruSeq Read 1 and TruSeq Read 2 as sequencing primer sites. In some instances, the cDNA library is sequenced using any method described herein, such that different sequencing domains specific to other sequencing methods and techniques can be incorporated into a capture probe or introduced during library preparation. In some instances, the sequence of the analyte or proxies thereof is determined via sequencing. In some instances, the sequencing is high-throughput sequencing. In some instances, the spatial barcode is sequenced, providing the location of the analyte.

### Kits

Also provided herein are kits including an array including a plurality of capture probes, where a capture probe of the plurality of capture probes includes a spatial barcode and a capture domain; and a bulky moiety coupled to a blocking reagent, wherein the blocking reagent inhibits the capture domain from hybridizing to a target analyte.

In some embodiments, the kit includes one or more fixatives. In some embodiments, the kit includes one or more biological stains. In some embodiments, the one or more biological stains includes hematoxylin and eosin.

In some embodiments, the kit includes one or more permeabilization reagent(s). In some kits, the one or more permeabilization reagent(s) is selected from the group consisting of an organic solvent, a cross-linking agent, a detergent, an enzyme, and combinations thereof.

In some embodiments, the kit includes a reverse transcriptase. In some embodiments, the kit includes a terminal deoxynucleotidyl transferase. In some embodiments, the kit includes a template switching oligonucleotide.

In some embodiments, the kit includes a DNA polymerase. In some embodiments, the kit includes a second strand primer. In some embodiments, the kit includes one or more adaptor(s). In some embodiments, the one or more adaptor(s) is/are selected from the group consisting of an i5 sample index sequence, an i7 sample index sequence, a P5 sample index sequence, a P7 sample index sequence, and combinations thereof.

Also provided herein are kits including an array including a plurality of capture probes, where a capture probe of the plurality of capture probes includes a spatial barcode and a capture domain; and a bulky moiety coupled to a blocking reagent, where the blocking reagent inhibits the capture domain from hybridizing to an analyte capture sequence.

In some embodiments, the kit includes a plurality of analyte capture agents, where an analyte capture agent of the plurality of analyte capture agents includes an analyte-binding moiety, an analyte-binding moiety barcode, and the analyte capture sequence. In some embodiments, the analyte-binding moiety barcode identifies the analyte-binding moiety. For example, the analyte-binding moiety barcode is a specific nucleotide sequence that identifies the analyte-binding moiety and therefore also identifies the analyte detected in the biological sample. In some embodiments, the analyte-binding moiety is an antibody, or a fragment thereof.

In some embodiments, the kit includes one or more fixative(s). In some embodiments, the kit includes one or more biological stains. In some embodiments, the one or more biological stains comprises hematoxylin and eosin.

In some embodiments, the kit includes one or more permeabilization reagent(s). In some embodiments, the one or more permeabilization reagent(s) is selected from the group consisting of an organic solvent, a cross-linking agent, a detergent, an enzyme, and combinations thereof.

In some embodiments, the kit includes a reverse transcriptase. In some embodiments, the kit includes a terminal deoxynucleotidyl transferase.

In some embodiments, the kit includes a template switching oligonucleotide. In some embodiments, the kit includes a DNA polymerase. In some embodiments, the kit includes a second strand primer.

In some embodiments, the kit includes one or more adaptor(s). In some embodiments, the one or more adaptor(s) is/are selected from the group consisting of an i5 sample index sequence, an i7 sample index sequence, a P5 sample index sequence, a P7 sample index sequence, and combinations thereof.

### Compositions

Also provided herein are compositions including an array having a first area, wherein the array comprises a plurality of capture probes, where the first area includes a capture probe of the plurality of capture probes including (i) a spatial barcode and (ii) a capture domain bound to a target analyte from a biological sample, where the biological sample was previously disposed on the first area and where the capture probe in the first area is not blocked by a blocking reagent, where the blocking reagent is coupled to a bulky moiety; and a second area of the array includes a capture probe of the plurality of capture probes including (i) a spatial barcode and (ii) a capture domain, where the blocking reagent inhibits the capture domain of the capture probe of the second area from hybridizing to the target analyte, and the second area is not covered by the biological sample and where the biological sample was previously disposed on the array.

Also provided herein are compositions including an array having a first area and a second area, where the array includes a plurality of capture probes, where: the first area includes a capture probe of the plurality of capture probes including (i) a spatial barcode and (ii) a capture domain bound to a target analyte from a biological sample,; and a second area of the array includes a capture probe of the plurality of capture probes including (i) a spatial barcode and (ii) a capture domain, where the capture probe of the plurality of capture probes in the second area is blocked by a blocking reagent coupled to a bulky moiety.

In some embodiments, the capture probe of the first area, the capture probe of the second area, or both, include one or more functional domains, a cleavage domain, a unique molecular identifier, and combinations thereof.

In some embodiments, the array includes one or more features. For example, the one or more features includes a bead, an inkjet spot, a masked spot, a well, or a hydrogel pad.

In some embodiments, the bulky moiety includes a bead. In some embodiments, the bead is a magnetic bead. In some embodiments, the bulky moiety includes a high molecular weight protein. In some embodiments, the bulky moiety is a polymer. In some embodiments, the polymer is polyethylene glycol (PEG). In some embodiments, the PEG has a molecular weight of about 1000 g/mol to about 20,000 g/mol. In some embodiments, the PEG has a molecular weight of at least about 1000 g/mol, at least about 2000 g/mol, at least about 3000 g/mol, at least about 4000 g/mol, at least about 5000 g/mol, at least about 6000 g/mol, at least about 7000 g/mol, at least about 8000 g/mol, at least about 9000 g/mol, at least about 10000 g/mol, at least about 11000 g/mol, at least about 12000 g/mol, at least about 13000 g/mol, at least about 14000 g/mol, at least about 15000 g/mol, at least about 16000 g/mol, at least about 17000 g/mol, at least about 18000 g/mol, at least about 19000 g/mol, or at least about 20,000 g/mol.

In some embodiments, the blocking reagent includes an enzyme. In some embodiments, the enzyme is a nuclease. In some embodiments, the nuclease is DNase. In some embodiments, the nuclease is uracil DNA glycosylase. For example, the blocking reagent coupled to the bulky moiety can be contacted with the second area of the array e.g., the area surrounding the biological sample. The bulky moiety keeps the blocking reagent in place and in some examples an enzyme is coupled to the bulky moiety. In such examples, the enzyme can be a nuclease (e.g., DNase) that degrades capture probes in the second area. In some examples, the enzyme can be a uracil DNA glycosylase capable of cleaving capture probes from the array in the second area. For example, the capture probe can include a cleavage domain where the cleavage domain includes one or more uracil bases where the uracil DNA glycosylase can cleave the capture probe thereby releasing the capture probe from the second area of the array and preventing unwanted binding outside the biological sample.

In some embodiments, the blocking reagent is a cleavage agent. In some embodiments, the cleavage agent is a chemical cleavage domain. Other examples of cleavage domains include labile chemical bonds such as, but not limited to, ester linkages (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), an abasic or apurinic/apyrimidinic (AP) site (e.g., cleavable with an alkali or an AP endonuclease), or a phosphodiester linkage (e.g., cleavable via a nuclease (e.g., DNAase)).

In some embodiments, the blocking reagent comprises a 3′ OH disabling agent. In some embodiments, the blocking reagent comprise a 3′ OH disabling agent. In some embodiments, the 3′ OH disabling agent is an exonuclease (e.g., any suitable exonuclease known in the art). In some embodiments, the exonuclease is a 3′ exonuclease. For example, a 3′ exonuclease can remove nucleotides from the 3′ hydroxyl terminus of the capture probe by cleavage of phosphodiester bonds via hydrolysis, thereby disabling the capture probe. In some embodiments, the 3′ OH disabling agent is a kinase. For example, the kinase (e.g., any suitable kinase known in the art) can phosphorylate the 3′ end of the capture probe, thereby disabling the capture probe from an extension reaction. In some embodiments, the 3′ OH disabling agent is a 3′ OH protecting group as described herein.

In some embodiments, the biological sample is a tissue sample. In some embodiments, the tissue sample is a fixed tissue sample. In some embodiments, the tissue sample is a fresh frozen tissue sample. In some embodiments, the biological sample is a tissue section. In some embodiments, the tissue section is a fresh frozen tissue section. In some embodiments, the tissue section is a fixed tissue section. In some embodiments, the fixed tissue section is a formalin-fixed paraffin-embedded tissue section, an acetone-fixed tissue section, a paraformaldehyde-fixed tissue section, or a methanol-fixed tissue section.

In some embodiments, the target analyte is nucleic acid. In some embodiments, the target nucleic acid is DNA. In some embodiments, the target nucleic acid is RNA. In some embodiments the RNA is mRNA.

Also provided herein are compositions, including an array having a first area, where the array comprises a plurality of capture probes, where: the first area includes a capture probe of the plurality of capture probes including (i) a spatial barcode and (ii) a capture domain bound to an analyte capture sequence, where the biological sample was previously disposed on the first area and where the capture probe in the first area is not blocked by a blocking reagent, where the blocking reagent is coupled to a bulky moiety; and a second area of the array includes a capture probe of the plurality of capture probes including (i) a spatial barcode and (ii) a capture domain, where the blocking reagent inhibits the capture domain of the capture probe of the second area from hybridizing to the analyte capture sequence, and the second area is not covered by the biological sample and where the biological sample was previously disposed on the array.

Also provided herein are compositions including an array having a first area and a second area, where the array includes a plurality of capture probes, where: the first area includes a capture probe of the plurality of capture probes including (i) a spatial barcode and (ii) a capture domain bound to an analyte capture sequence; and a second area of the array includes a capture probe of the plurality of capture probes including (i) a spatial barcode and (ii) a capture domain, where the capture probe of the plurality of capture in the second area is blocked by a blocking reagent coupled to a bulky moiety.

In some embodiments, the composition includes a plurality of analyte capture agents, where an analyte capture agent of the plurality of analyte capture agents includes an analyte-binding moiety, an analyte-binding moiety barcode, and the analyte capture sequence. In some embodiments, the analyte-binding moiety barcode identifies the analyte-binding moiety. In some embodiments, the analyte-binding moiety is an antibody, or a fragment thereof.

In some embodiments, the capture probe of the first area, the capture probe of the second area, or both, include one or more functional domains, a cleavage domain, a unique molecular identifier, and combinations thereof.

In some embodiments, the array includes one or more features. For example, the one or more features includes a bead, an inkjet spot, a masked spot, a well, or a hydrogel pad.

In some embodiments, the bulky moiety includes a bead. In some embodiments, the bead is a magnetic bead. In some embodiments, the bulky moiety includes a high molecular weight protein. In some embodiments, the bulky moiety is a polymer. In some embodiments, the polymer is polyethylene glycol (PEG). In some embodiments, the PEG has a molecular weight of about 1000 g/mol to about 20,000 g/mol. In some embodiments, the PEG has a molecular weight of at least about 1000 g/mol, at least about 2000 g/mol, at least about 3000 g/mol, at least about 4000 g/mol, at least about 5000 g/mol, at least about 6000 g/mol, at least about 7000 g/mol, at least about 8000 g/mol, at least about 9000 g/mol, at least about 10000 g/mol, at least about 11000 g/mol, at least about 12000 g/mol, at least about 13000 g/mol, at least about 14000 g/mol, at least about 15000 g/mol, at least about 16000 g/mol, at least about 17000 g/mol, at least about 18000 g/mol, at least about 19000 g/mol, or at least about 20,000 g/mol.

In some embodiments, the blocking reagent includes an enzyme. In some embodiments, the enzyme is a nuclease. In some embodiments, the nuclease is DNase. In some embodiments, the nuclease is uracil DNA glycosylase. For example, the blocking reagent coupled to the bulky moiety can be contacted with the second area of the array e.g., the area surrounding the biological sample. The bulky moiety keeps the blocking reagent in place and in some examples an enzyme is coupled to the bulky moiety. In such examples, the enzyme can be a nuclease (e.g., DNase) that degrades capture probes in the second area. In some examples, the enzyme can be a uracil DNA glycosylase capable of cleaving capture probes from the array in the second area. For example, the capture probe can include a cleavage domain where the cleavage domain includes one or more uracil bases where the uracil DNA glycosylase can cleave the capture probe thereby releasing the capture probe from the second area of the array and preventing unwanted binding outside the biological sample.

In some embodiments, the blocking reagent comprise a 3′ OH disabling agent. In some embodiments, the 3′ OH disabling agent is an exonuclease (e.g., any suitable exonuclease known in the art). In some embodiments, the exonuclease is a 3′ exonuclease. For example, a 3′ exonuclease can remove nucleotides from the 3′ hydroxyl terminus of the capture probe by cleavage of phosphodiester bonds via hydrolysis, thereby disabling the capture probe. In some embodiments, the 3′ OH disabling agent is a kinase. For example, the kinase (e.g., any suitable kinase known in the art) can phosphorylate the 3′ end of the capture probe, thereby disabling the capture probe from an extension reaction. In some embodiments, the 3′ OH disabling agent is a 3′ OH protecting group as described herein.

In some embodiments, the blocking reagent includes a chemical cleavage agent. In some embodiments, the cleavage agent is a chemical cleavage domain. Other examples of cleavage domains include labile chemical bonds such as, but not limited to, ester linkages (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), an abasic or apurinic/apyrimidinic (AP) site (e.g., cleavable with an alkali or an AP endonuclease), or a phosphodiester linkage (e.g., cleavable via a nuclease (e.g., DNAase)).

In some embodiments, the biological sample is a tissue sample. In some embodiments, the tissue sample is a fixed tissue sample. In some embodiments, the tissue sample is a fresh frozen tissue sample. In some embodiments, the biological sample is a tissue section. In some embodiments, the tissue section is a fresh frozen tissue section. In some embodiments, the tissue section is a fixed tissue section. In some embodiments, the fixed tissue section is a formalin-fixed paraffin-embedded tissue section, an acetone-fixed tissue section, a paraformaldehyde-fixed tissue section, or a methanol-fixed tissue section. In some embodiments, the analyte-binding moiety binds a protein.

### EXAMPLES

### Example 1

### Reducing Capture of Analytes in Areas of the Array Not Covered by the Biological Sample

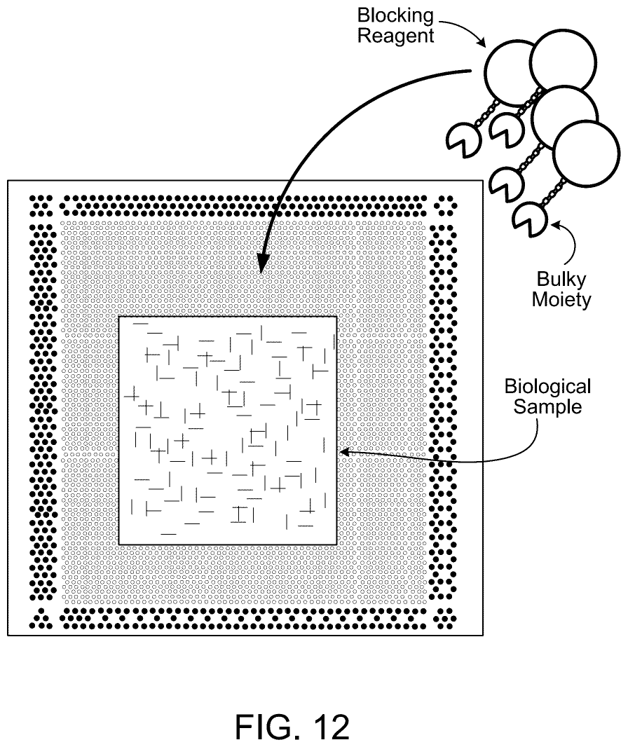

As described herein FIG. 5 is a schematic diagram showing an exemplary capture probe. The capture probe can include a cleavage domain, one or more functional sequences, a unique molecular identifier, a spatial barcode, and a capture domain. The capture domain facilitates capture of a target analyte (e.g., nucleic acid) or a proxy of an analyte such as a ligation product or an analyte capture sequence of an analyte capture agent. FIG. 12 shows a biological sample on an array, however, there are portions of the array that are not covered by the biological sample. When the biological sample is permeabilized to release the analytes and allow capture of said analytes on the array, some analyte can diffuse to areas outside the biological sample. Analytes captured outside the area covered by the biological sample can waste resources and generate inaccurate data. FIG. 12 also shows a bulky moiety (e.g., a high-molecular weight protein, a polymer (e.g., PEG), etc.) coupled to a blocking reagent (e.g., an enzyme, a 3′ OH disabling agent, a chemical cleavage agent, etc.). The bulky moiety and blocking reagent are contacted with the array (e.g., contacted with areas of the array not covered by the biological sample) where the bulky moiety keeps the blocking reagent in the position in which it was deposited. Depending on the blocking reagent various ethods of reducing capture of analytes in the area not covered by the biological sample include cleaving the capture probe from the array, either enzymatically or chemically, degrading the capture probe (e.g., with a DNase enzyme), or with a 3′ OH disabling agent. These methods reduce capture of analytes in areas of the array not covered by the biological sample, thus avoiding wasted resources and generating accurate data.

What is claimed is:

1. A method comprising:
(a) disposing a biological sample on an array at a first area, wherein the array comprises a plurality of capture probes, wherein:
(i) the first area comprises a capture probe of the plurality of capture probes comprising: (i) a spatial barcode and (ii) a capture domain, and
(ii) a second area comprises a capture probe of the plurality of capture probes comprising: (i) a spatial barcode and (ii) a capture domain, and the second area is an area of the array not covered by the biological sample disposed on the array;
(b) contacting the second area of the array with a bulky moiety coupled to a blocking reagent, wherein the blocking reagent inhibits the capture probe in the second area from hybridizing to a target analyte; and
(c) hybridizing the capture domain of the capture probe of the first area to the target analyte.

2. The method of claim 1, wherein the capture probe of the first area, the capture probe of the second area, or both, further comprise one or more functional domains, a cleavage domain, a unique molecular identifier, and combinations thereof.

3. The method of claim 1, wherein the array comprises one or more features, wherein the one or more features comprises a bead, an inkjet spot, a masked spot, a well, or a hydrogel pad.

4. The method of claim 1, wherein the bulky moiety comprises a magnetic bead, a high molecular weight protein, or a polymer.

5. The method of claim 4, wherein the polymer comprises a polyethylene glycol (PEG) having a molecular weight of about 3000 g/mol to about 10,000 g/mol.

6. The method of claim 1, wherein the blocking reagent comprises an enzyme.

7. The method of claim 6, wherein the enzyme is a nuclease, wherein the nuclease is one or more of a DNase or a uracil DNA glycosylase.

8. The method of claim 1, wherein the blocking reagent comprises a 3′ OH disabling agent or a chemical cleavage agent.

9. The method of claim 1, wherein the method further comprises, prior to step (b), fixing the biological sample wherein the step of fixing the biological sample comprises the use of a fixative selected from the group consisting of: ethanol, methanol, acetone, formaldehyde, paraformaldehyde-Triton, glutaraldehyde, and combinations thereof.

10. The method claim 1, wherein the method further comprises, prior to step (b), staining the biological sample,
wherein the step of staining the biological sample comprises the use of a biological stain selected from the group consisting of: acridine orange, Bismarck brown, carmine, Coomassie blue, cresyl violet, 4′,6-diamidino-2-phenylindole (DAPI), eosin, ethidium bromide, acid fuchsine, hematoxylin and eosin, Hoechst stains, iodine, methyl green, methylene blue, neutral red, Nile blue, Nile red, osmium tetroxide, propidium iodide, rhodamine, safranin, and combinations thereof, or
wherein the step of staining the biological sample comprises the use of a detectable label selected from the group consisting of: a radioisotope, a fluorophore, a chemiluminescent compound, a bioluminescent compound, and combinations thereof.

11. The method of claim 1, wherein the biological sample is a tissue sample.

12. The method of claim 11, wherein the tissue sample is a fixed tissue sample or a fresh frozen tissue sample.

13. The method of claim 1, wherein the biological sample is a tissue section, optionally wherein the tissue section is fixed tissue section or a fresh-frozen tissue section.

14. The method of claim 13, wherein the fixed tissue section is a formalin-fixed paraffin-embedded tissue section, an acetone-fixed tissue section, a paraformaldehyde-fixed tissue section, or a methanol-fixed tissue section.

15. The method of claim 1, further comprising (d) determining (i) a sequence corresponding to the spatial barcode of the capture probe of the first area of the array, or a complement thereof, and (ii) all or a portion of a sequence corresponding to the target analyte, or a complement thereof, and using the sequences of (i) and (ii) to determine a location of the target analyte in the biological sample.

16. The method of claim 15, wherein the determining in (d) comprises sequencing (i) the spatial barcode of the capture probe of the first area of the array, or the complement thereof, and (ii) all or a portion of the target analyte, or the complement thereof.

17. The method of claim 1, further comprising extending a 3′ end of the capture probe of the first area of the array using the target analyte as a template, thereby generating an extended capture probe and optionally, generating a second strand complementary to the extended capture probe.

18. The method of claim 1, wherein the method further comprises imaging the biological sample.

19. The method of claim 1, wherein the target analyte is genomic DNA or mRNA.

20. The method of claim 1, further comprising permeabilizing the biological sample.
